(19) United States
(12) Patent Application Publication
(10) Pub. No.: US 2005/0123577 A1
(43) Pub. Date: Jun. 9, 2005

(54) Use of prostaglandin EP-3 receptor antagonists as cosmetic agents for attenuating, reducing or stopping the loss of head hair and other hairs

(75) Inventors: Jean Francois Michelet, Creteil (FR); Yann Mahe, Morsang-Sur-Orge (FR); Bruno Bernard, Neuilly-Sur-Seine (FR)

Correspondence Address: JACOBSON HOLMAN PLLC, 400 SEVENTH STREET N.W., SUITE 600, WASHINGTON, DC, 20004, US

(73) Assignee: L'oreal, Paris (FR)

(21) Appl. No.: 11/035,053

(22) Filed: Jan. 14, 2005

Related U.S. Application Data
(62) Division of application No. 09/917,215, filed on Jul. 30, 2001.

(30) Foreign Application Priority Data
Jul. 28, 2000 (FR) 00 09985

(51) Int. Cl. A61K 31/557; A61K 7/00

(52) U.S. Cl. 424/401; 514/573

(57) ABSTRACT

The invention relates to the use of prostaglandin EP-3 receptor antagonists as cosmetic agents for attenuating, reducing or stopping the loss of head hair and other hairs.

The invention relates to the use of prostaglandin EP-3 receptor antagonists as cosmetic agents for attenuating, reducing or stopping loss of head hair and other hairs.

Man has a complement of 100 000 to 150 000 hairs and it is normal to lose 50 to 100 hairs daily. Maintenance of this complement results essentially from the fact that the life of a hair is subject to a hair cycle in the course of which the hair forms, grows and falls out, before being replaced with a new hair which appears in the same follicle.

Three phases are observed in the course of a hair cycle, namely: the anagenic phase, the catagenic phase and the telogenic phase.

In the course of the first phase, known as the anagenic phase, the hair passes through a period of active growth associated with intense mitotic activity at the bulb.

The second phase, known as the catagenic phase, is transient and is marked by an interruption of the mitotic activity of the bulb. During this phase, the hair undergoes an involution, the follicle becomes atrophied and its dermal implantation moves upwards.

The end phase, known as the telogenic phase, corresponds to a resting period of the follicle and the hair finishes by falling out. After this resting phase, a new follicle is regenerated, in the place of the previous one.

This process of permanent physical renewal undergoes a natural evolution in the course of ageing, the hairs become finer and their cycles shorter (M. Courtois et al., 1995, Br. J. Dermatol., 132: 86-93).

In almost all cases, hair loss occurs in genetically predisposed individuals; it more particularly affects men.

This hair loss occurs when the process of physical renewal is accelerated or disrupted, i.e. the growth phases are shortened (Mr Courtois et al., 1994, Skin Pharmacol., 7: 84-89), the hairs pass to the telogenic phase earlier and they fall out in larger numbers. The successive growth cycles result in increasingly fine and increasingly short hairs, which become converted gradually into an unpigmented down. This phenomenon may lead to baldness.

Compositions for preventing or reducing hair loss and especially for inducing or stimulating hair growth have been sought for many years in the cosmetic or pharmaceutical industry.

In this perspective, compounds such as 6-1-(piperidyl)-2,4-pyrimidinediamine 3-oxide or <<Minoxidil>> have been used. The use of a lotion containing an azole derivative and most specifically 1-acetyl-4-(4-[2-(2,4-di-chlorophenyl)-2-(1H-imidazol-1-ylmethyl)-1,3-dioxolan-4-ylmethoxy]phenyl)piperazine for the treatment of alopecia is described in patent WO 92/00057.

In parallel, the article <<Growth regulation of primary human keratinocytes by prostaglandin E receptor EP₂ and EP₃ subtypes>> by Konger et al. (Biochimica Biophysica Acta, 1401, 1998, 221-224) describes that prostaglandin EP-3 receptors play an important role in regulating the growth of epidermal keratinocytes.

Nevertheless, it is well known that the programmes of differentiation of the keratinocytes of the epidermis and of hair follicles are clearly different. Thus, it is known that differentiation markers such as keratins K1 and K10 are not expressed in hair follicles and in particular in the outer sheath (Lenoir et al., 1988, Dev. Biol. 130: 610-620); that trichohyalin is expressed in hair follicles, in particular in the inner sheath but not in the epidermis (O'Guin et al., 1992, J. Invest. Dermatol. 98: 24-32); and that type 1 cyclooxygenase is not expressed in the keratinocytes of hair follicles but is expressed in the epidermis (Michelet et al., 1997, J. Invest. Dermatol. 108: 205-209).

Furthermore, it is known that the keratinocytes of the epidermis and of hair follicles behave differently in response to the same pharmacological agent. Thus, it is known that, in vivo, treating the epidermis with retinoic acid induces hyperplasia and spongiosis (Griffiths et al., 1993, J. Invest. Dermatol. 101: 325-328) whereas treating the scalp induces a loss of hair (Berth-Jones et al., 1990, Br. J. Dermatol. 122: 75-755), and that, in vitro, retinoic acid, depending on the dose used, promotes or reduces the differentiation of the epidermis (Asselineau et al., 1989, Dev. Biol. 133: 32-335), while it causes an interruption of growth of the hair follicles (Billoni et al., 1997, Acta Dermatol. Venerol. 77: 350-355). It is also known that EGF induces epidermal hyperplasia and, simultaneously, regression of the hair follicles (Philip et al., 1985, J. Invest. Dermatol. 84: 172-175).

Moreover, it is known that sulprostone is a prostaglandin EP-3 receptor agonist, in document <<VII. International Union of Pharmacology Classification of Prostanoid Receptors: Properties, Distribution, and Structure of the Receptors and Their Subtypes>> by Coleman et al., Pharmacological Reviews, 1994, Vol. 46. The said document points out that prostaglandin receptors induce a reduction in the level of cAMP. An example of an EP-3 receptor antagonist is, for example, the non-prostanoic antagonist L789106 described by MERCK FROOST (M. Labelle et al., 11th International Conference on Advances in Prostaglandin and leukotriene research: Basic science and new clinical applications. Florence, from 4 to 8 June 2000).

Prostaglandins are biological effectors derived from polyunsaturated fatty acids such as, for example, arachidonic acid for PGA₂, PGE₂, PGF_{2α} and TXA₂, or dihomo-γ-linolenic acid for PGE₁. Prostaglandins are involved in many physiological regulation phenomena.

The Applicant has now discovered that by using prostaglandin EP-3 receptor antagonists, a large induction and large stimulation in the growth of head hair and other hairs and strong action on slowing down the loss of head hair and other hairs are found, surprisingly.

The Applicant has thus found that the use in accordance with the invention makes it possible to obtain a rapid effect, at a low concentration and/or with a low rate of application. These antagonists are capable of stopping the loss of head hair and other hairs and/or of increasing the regrowth of head hair and other hairs.

Furthermore, the prostaglandin EP-3 receptor antagonists of the invention are particularly of low toxicity and show good conservation.

The use of these antagonists makes it possible to obtain, in particular compared with those known beforehand, more effective compositions which may be used in particularly easy manner, and which also allow the compositions to be removed easily by simple rinsing.

The compounds in accordance with the invention are moreover particularly suitable in cosmetic terms and do not cause any irritation of the scalp, even after prolonged contact, without rinsing.

Thus, one subject of the invention is the use of prostaglandin EP-3 receptor antagonists as a cosmetic or dermatological agents for attenuating, reducing or stopping the loss of head hair and other hairs. These antagonists are also capable of increasing the regrowth of head hair and other hairs.

A subject of the invention is also the use of a prostaglandin EP-3 receptor antagonist in a cosmetic composition and also in a cosmetic treatment process for attenuating, reducing or stopping the loss of head hair and other hairs.

The main subject of the invention is a cosmetic or dermatological composition containing at least one prostaglandin EP-3 receptor antagonist in a cosmetically or dermatologically acceptable medium.

The prostaglandin EP-2 and/or EP-4 receptors are receptors of prostaglandins of the E2 series. These receptors combine a family of 4 major representatives (EP1, EP2, EP3 and EP4) and have very varied tissue activities.

An antagonist is a compound which binds to a receptor and which does not induce a biological responce, unlike the agonist which induces a response similar to that obtained with the natural ligand which activates this response.

The expression <prostaglandin EP-3 receptor antagonists means antagonist compounds which make it possible to attenuate, reduce or stop the loss of head hair and other hairs. These antagonists are capable of stopping the loss of head hair and other hairs and/or increase the regrowth of head hair and other hairs.

The term <<other hairs>> also means the eyelashes, the eyebrows and any hairs in general.

According to the invention, the said cosmetic composition may contain from 0.001% to 10% and preferably from 0.1% to 5% of prostaglandin EP-3 receptor antagonists by weight relative to the weight of the composition.

It is also possible to use in addition other cosmetic or dermatological agents for stopping the loss of head hair and other hairs and/or increasing the growth of head hair and other hairs in the cosmetic compositions defined above, such as, for example, prostaglandin EP-2 and/or receptor agonists in proportions ranging from 0.001% to 10% and preferably from 0.1% to 5% of agonists by weight relative to the weight of the composition, or alternatively compounds known for their properties on the loss and/or growth of head hair and/or other hairs, such as, for example, Minoxidil or 2,4-diaminopyrimidine 3-oxide or Aminexil.

The physiologically acceptable medium used for the compositions of the invention is a medium which can consist of water or a mixture of water and a solvent or a mixture of solvents. The solvents are chosen from acceptable organic solvents chosen more particularly from C1-C4 lower monofunctional or polyfunctional alcohols, for instance ethanol, isopropanol, tert-butanol, optionally oxyethylenated polyethylene glycols, polypropylene glycol esters, sorbitol and its derivatives, dialkyl isosorbides, glycol ethers and propylene glycol ethers, and fatty esters.

When they are present, the solvents are present in proportions of between 5% and 98% by weight relative to the total weight of the composition.

The composition may in addition contain a fatty phase. In this case, the fatty phase represents 0% to 50% of the total weight of the composition.

These compositions may also contain:
esterified oligosaccharides such as those described in EP-A-0 064 012;
hexosaccharic acid derivatives such as those described in EP-A-0 375 388, in particular glucosaccharic acid;
glycosidase inhibitors such as those described in EP-A-0 334 586, in particular D-glycaro-1,5-lactam;
glycosaminoglycanase and proteoglycanase inhibitors such as those mentioned in EP-A-0 277 428, in particular L-galactano-1,4-lactone;
tyrosine kinase inhibitors such as those described in EP-A-0 403 238, in particular 1-amido-1-cyano-(3,4-dihydroxyphenyl)ethylene;
hyperaemiants such as:
   nicotinic acid esters including, more particularly, benzyl and C₁-C₆ alkyl nicotinates and in particular methyl and benzyl nicotinate, and also tocopheryl nicotinate;
   xanthine bases including, more particularly, caffeine and theophylline;
   capsaicin;
UV-A and UV-B screening agents, for instance methoxycinnamates and benzophenone derivatives;
phosphodiesterase inhibitors such as Visnadine®;
adenine cyclase activators such as Forskolin;
antioxidants and free-radical scavengers, in particular
   for OH radicals such as DMSO;
   α-tocopherol, BHA and BHT;
   superoxide dismutase (SODIUM);
antidandruff agents such as omadine and octopirox;
moisturizers such as urea, glycerol, lactic acid, α-hydroxy acids, thiamorpholinone and its derivatives, and lactones;
antiseborrhoeic agents such as S-carboxymethylcysteine, S-benzylcysteamine and derivatives thereof, and thioxolone;
antiandrogens and hormones such as oesrtiol, oestradiol, thyroxine, oxendolone and diethylstilbestrol;
retinoids including, more particularly, t-trans-retinoic acid, also known as tretinoin, isotretinoin, retinol or vitamin A and its derivatives, such as the acetate, palmitate or propionate, motretinide, etretinate and zinc t-trans-retinoate;
antibacterial agents chosen, more particularly, from Irgasan, macrolides, pyranosides and tetracyclines, and in particular erythromycin;
calcium antagonists, among which mention may be made of Cinnarizine and Diltiazem as non-limiting examples;
phospholipids such as lecithin;
diazoxide (3-methyl-7-chloro-1,2,4-[2H]benzothiadiazine 1,1-dioxide);
linoleic and linolenic acids;
anthralin and its derivatives;
5-alkanol salicylic acid and its derivatives as described in patent FR-2 581 542;
penetration activators such as THF, 1,4-dioxane, oleic acid, 2-pyrrolidone, benzyl salicylate, etc.,
vitamins or provitamins such as β-carotene, biotin, panthenol and its derivatives, vitamin C and vitamins B₂, B₄ and B₆.

These compositions may also contain cyclic AMP.

These compositions may also additionally contain preserving agents, stabilizers, pH regulators, osmotic pressure modifiers, emulsifiers and conventional hydrophilic or lipophilic gelling agents and/or thickeners; hydrophilic or lipophilic active agents; preserving agents; antioxidants; fragrances; emulsifiers; moisturizers; pigmenting agents; depigmenting agents; keratolytic agents; vitamins; emollients; sequestering agents; surfactants; polymers; acidifying or basifying agents; fillers; free-radical scavengers; ceramides; sunscreens; insect repellents; slimming agents; dyestuffs; bactericides; antidandruff agents.

The compositions in accordance with the invention may also contain surfactants including, in particular, those chosen from nonionic and amphoteric surfactants.

Among the nonionic surfactants, those which will be mentioned are the polyhydroxypropyl ethers described in particular in French patents Nos. 1 477 048; 2 091 516; 2 169 787; 2 328 763; 2 574 786; oxyethylenated (C₈-C₉)alkylphenols comprising from 1 to 100 mol of ethylene oxide and preferably 5 to 35 mol of ethylene oxide; alkylpolyglycosides of formula: CₙH₂ₙ₊₁ (C₆H₁₀O₅)ₓH in which n ranges from 8 to 15 inclusive and x from 1 to 10 inclusive.

Among the amphoteric surfactants, those which will be mentioned more particularly are the amphocarboxyglycinates and amphocarboxypropionates defined in the CTFA dictionary, 3rd edition, 1982, and sold in particular under the name Miranol® by the company Miranol.

Cationic and/or anionic surfactants may also be used.

The compounds in accordance with the invention may also be introduced into gelled or thickened supports, such as essentially aqueous supports gelled with hetero-biopolysaccharides, such as xanthan gum, scleroglucans or cellulose derivatives, in particular cellulose ethers, aqueous-alcoholic supports gelled with polyhydroxyethyl acrylates or methacrylates or essentially aqueous supports thickened in particular with polyacrylic acids crosslinked with a polyfunctional agent, such as the Carbopols sold by the company Goodrich.

The thickeners are preferably present in proportions of between 0.05% and 5% by weight and in particular between 0.2% and 3% by weight relative to the total weight of the composition.

Needless to say, a person skilled in the art will take care to select the optional compound(s) to be added to the composition according to the invention, such that the advantageous properties intrinsically associated with the composition in accordance with the invention are not, or are not substantially, adversely affected by the addition envisaged.

The composition defined above may be in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules.

The composition may have a pH of between 3 and 8.

The composition may have the appearance of a white or coloured cream, an ointment, a milk, a lotion, a serum, a paste, a mousse or a solid.

These compositions defined above may be applied to the hair or the scalp and can be applied, for example, after washing the scalp and the hair with a shampoo.

A subject of the invention is also the use of prostaglandin EP-3 receptor antagonists as cosmetic or dermatological agents for attenuating, reducing or stopping the loss of head hair and other hairs. These antagonists are also capable of increasing the regrowth of head hair and other hairs.

A subject of the invention is also the use of a composition as defined above to attenuate, reduce or stop loss of head hair and other hairs.

Another subject of the invention is a cosmetic or dermatological treatment process for attenuating, reducing or stopping the loss of head hair and other hairs, which consists in applying to head hair or other hairs a cosmetically or dermatologically effective amount of prostaglandin EP-3 receptor antagonists.

Another cosmetic or dermatological treatment process for attenuating, reducing or stopping the loss of head hair and other hairs consists in applying to head hair or other hairs a cosmetic or dermatological composition as defined above.

The examples which follow are intended to illustrate the invention without, however, being limiting in nature.

### EXAMPLE 1

### Lotion for Preventing Hair Loss

| | | |
|---|---|---|
| Prostaglandin EP-3 receptor antagonist | 0.5 | g |
| Propylene glycol | 20 | g |
| 95° Ethanol | 30 | g |
| Water qs | 100 | g |

This lotion is applied daily at a rate of 10 ml to the scalp for 2 to 3 months. A marked slowing down in the daily loss of head hair and other hairs is then observed.

### EXAMPLE II

### Shampoo for Preventing Hair Loss

| | | |
|---|---|---|
| Prostaglandin EP-3 receptor antagonist | 1.5 | g |
| Polyglyceryl 3-hydroxylauryl ether | 26 | g A.M. |
| Hydroxypropylcellulose sold under the | 2 | g |
| name Klucell G by the company Hercules | | |

| | |
|---|---|
| Preserving agent | qs |

Prostaglandin EP-2 and/or EP-4 Receptor

| | | |
|---|---|---|
| agonist | 1 | g |
| 95° Ethanol | 50 | g |
| Aminexil | 0.1 | g |
| Water qs | 100 | g |

This shampoo is used daily at a rate of 15 g per head of hair, with an exposure time of about one minute, over a period of 4 months. An appreciable slowing down in the daily loss of hair is then observed.

### EXAMPLE III

### Gel for Preventing Hair Loss

| Prostaglandin EP-3 receptor antagonist | 0.75 | g |
|---|---|---|
| Essential oil of eucalyptus | 1 | g |
| Econazole | 0.25 | g |
| Lauryl polyglyceryl 6 cetearyl glycol | 1.9 | g |
| ether | | |
| Sodium glutamate of hydrogenated tallow, | 0.1 | g |
| sold under the name Acylglutamate HS110 | | |
| by the company Ajinomoto | | |
| Preserving agents | qs | |
| Carbopol 934P sold by the company BF | 0.3 | g A.M. |
| Goodrich Corporation | | |
| Neutralizer | qs pH 7 | |
| Water qs | 100 | g |

This gel is applied twice a day (morning and evening) at a rate of 25 g to the entire scalp with final massaging. After application for 3 months, the daily loss of head hair and other hair is clearly slowed down.

### EXAMPLE IV

### Lotion for Preventing Hair Loss

| | | |
|---|---|---|
| Prostaglandin EP-3 receptor antagonist | 0.4 | g |
| Propylene glycol | 20 | g |
| 95° Ethanol | 50 | g |
| Aminexil | 0.1 | g |
| Water qs | 100 | g |

This lotion is used in the same way as in Example 1. The results observed are of the same order.

Experiment:

In order to study the behaviour of hair follicles in the presence of prostaglandin EP-3 receptor antagonists, the Applicant used the <<surviving hair>> method from L'Oréal patent FR 9508465.

From a scalp biopsy, a fairly thin strip of scalp was isolated using a scalpel. With microtweezers, the adipose tissue around the follicles was removed, while taking care not to damage the hair bulb. Under a microscope, the follicle was cut away using a scalpel to separate it from its epidermal and dermal environment.

One of the fragments obtained was cultured in Williams E medium at 37° C. under a humid atmosphere in the presence of 5% CO₂ and was used as control.

The other fragments were placed in the same culture medium in the presence of prostaglandin EP-3 receptor antagonists.

The fragments in the presence of the agonists thus maintained in cell culture extend in a significantly greater manner in comparison with the antagonist-free control fragment.

1. A cosmetic composition comprising at least one prostaglandin EP-3 receptor antagonist in a cosmetically acceptable medium.

2. The composition according to claim 1, wherein the composition comprises from 0.001% to 10% of the antagonist by weight relative to the weight of the composition.

3. The composition according to claim 1, and further comprising from 0.001% to 10% of a prostaglandin EP-2 receptor agonist, a prostaglandin EP-4 receptor agonist, or a combination thereof by weight relative to the weight of the composition.

4. The composition according to claim 1, and further comprising a cosmetically acceptable medium including water or mixtures of water and at least one organic solvent selected from the group consisting of hydrophilic organic solvents, lipophilic organic solvents, amphiphilic organic solvents, and mixtures thereof.

5. The composition according to claim 4, wherein the organic solvent is selected from the group consisting of monofunctional or polyfunctional alcohols, oxyethylenated polyethylene glycols, polypropylene glycol esters, sorbitol and derivatives thereof, dialkyl isosorbides, glycol ethers, polypropylene glycol ethers, and fatty esters.

6. The composition according to claim 4, wherein the organic solvent is present in an amount of 5% to 98% of the total weight of the composition.

7. The composition according to claim 1, and further comprising at least one fatty phase.

8. The composition according to claim 7, wherein the fatty phase is present in an amount of 50% or less of the total weight of the composition.

9. The composition according to claim 1, and further comprising at least one additive selected from the group consisting of: conventional hydrophilic or lipophilic gelling agents; conventional hydrophilic or lipophilic thickeners; hydrophilic or lipophilic active agents; preserving agents; antioxidants; fragrances; emulsifiers; moisturizers; pigmenting agents; depigmenting agents; keratolytic agents; vitamins; emollients; sequestering agents; surfactants; polymers; acidifying or basifying agents; fillers; free-radical scavengers; ceramides; sunscreens; insect repellents; slimming agents; dyestuffs; bactericides; and antidandruff agents.

10. The composition according to claim 1, wherein the composition is an aqueous solution, an aqueous-alcoholic solution, an oily solution, an oil-in-water emulsion, a water-in-oil emulsion, a multiple emulsion, an aqueous gel, an oily gel, a liquid anhydrous product, a pasty anhydrous product, a solid anhydrous product, or a dispersion of oil in an aqueous phase using spherules.

11. The composition according to claim 1, wherein the composition has an appearance of a white cream, a colored cream, an ointment, a milk, a lotion, a serum, a paste, a mousse, or a solid.

12. The composition according to claim 1, wherein the composition has a pH of between 3 and 8.

13. A method for attenuating or reducing the loss of keratin materials in a subject which comprises administering to the subject a prostaglandin EP-3 receptor antagonist.

14. A method for attenuating or reducing the loss of hair in a subject which comprises administering to the subject the composition of claim 1.

15. A cosmetic treatment process for attenuating or reducing the loss of hair which comprises applying to the hair a cosmetically effective amount of a prostaglandin EP-3 receptor antagonist.

16. A cosmetic treatment process for attenuating or reducing the loss of hair which comprises applying to the hair the cosmetic composition of claim 1.

17. The composition according to claim 1, wherein the composition comprises from 0.1 % to 5% of the antagonist by weight relative to the weight of the composition.

18. The composition according to claim 1, and further comprising from 0.1% to 5% of aprostaglandin EP-2 receptor agonist, a prostaglandin EP-4 receptor agonist, or a combination thereof by weight relative to the weight of the composition.

19. The method according to claim 14, wherein the hair is head hair.

20. The composition according to claim 1, and further comprising at least one compound known for its properties on the loss of hair or growth of hair.

21. The composition according to claim 20, wherein the compound known for its properties on the loss of hair or growth of hair is selected from the group consisting of Minoxidil, 2,4-diaminopyrimidine 3-oxide, and Aminexil.

22. The composition according to claim 1, and further comprising cyclic AMP.

23. The composition according to claim 1, in a cosmetically acceptable gelled or thickened medium having thickeners.

24. The composition according to claim 23, wherein the gelled or thickened medium is selected from the group consisting of aqueous medium gelled with heterobiopolysaccharides, aqueous-alcoholic medium gelled with polyhydroxyethylacrylates or methacrylates, and aqueous medium thickened with polyacrylic acids crosslinked with a polyfunctional agent.

25. The composition according to claim 23, wherein the thickeners are present in proportions of between 0.05% and 5% by weight relative to the total weight of the composition.

26. The composition according to claim 23, wherein the thickeners are present in proportions of between 0.2% and 3% by weight relative to the total weight of the composition.
